(19) United States
(12) Patent Application Publication
(10) Pub. No.: US 2026/0229336 A1
(43) Pub. Date: Aug. 6, 2026

(54) DIGITAL THERAPEUTICS AND COMBINATION THERAPY FOR ENHANCING FERTILITY

(71) Applicant: REMEPY HEALTH LTD, Ramat Hasharon (IL)

(72) Inventors: AMIR AMEDI, Modiin-Macabim Reut (IL); MICHAL TSUR-SHALEV, Zichron Yaacov (IL); OR SHOVAL, RAMAT HASHARON (IL); NIRA NEOMI SAPORTA, GIVATAYIM (IL); RENAN GUTMAN, Hoboken, NJ (US)

(73) Assignee: REMEPY HEALTH LTD, Ramat Hasharon (IL)

(21) Appl. No.: 19/449,511

(22) Filed: Jan. 15, 2026

Related U.S. Application Data
(60) Provisional application No. 63/745,360, filed on Jan. 15, 2025.

(51) Int. Cl. G16H 20/30; G16H 10/60; G16H 20/10; G16H 20/60; G16H 20/70

(52) U.S. Cl. CPC G16H 20/30; G16H 10/60; G16H 20/10; G16H 20/60; G16H 20/70

(57) ABSTRACT

Provided are methods for improving reproductive health and outcomes in women through personalized, technology-enabled therapeutic plans. The invention integrates physical activity, psychological support, and nutritional management into a comprehensive program delivered via digital platforms. The platforms utilizes dynamic algorithms to adapt interventions to a woman's profile, enabling real-time adjustments throughout implementation of the interventions.

### FIELD OF THE INVENTION

The present invention relates to methods and systems for improving reproductive health and outcomes in women. More specifically, it pertains to the use of personalized, technology-enabled therapeutic plans that integrate physical activity, psychological support, and nutritional management, delivered through digital platforms. By leveraging these platforms, the invention provides dynamic and adaptive interventions that are tailored to individual needs, ensuring a comprehensive approach to reproductive wellness.

### BACKGROUND

Infertility and suboptimal reproductive outcomes remain significant challenges for women worldwide, affecting physical health, emotional well-being, and quality of life. Despite advances in assisted reproductive technologies and medical interventions, many existing solutions fail to address the multifactorial nature of fertility, which is influenced by physical, psychological, and nutritional factors. Current approaches often focus narrowly on clinical procedures, overlooking the importance of holistic lifestyle management and personalized support, and further overlooking the need to synchronize such support with time-sensitive fertility treatment phases and medication schedules.

The present invention provides an integrative solution that bridges this gap by delivering a comprehensive therapeutic plan through a personal electronic device. This plan combines physical activity interventions, psychological support strategies, and nutrition management, ensuring a tailored and dynamic approach to improving reproductive outcomes. By transforming comprehensive therapeutic plans into tailored, interactive outputs and monitoring user engagement and progress over time, the approach enables adaptive, data-driven guidance that evolves with individual needs. This solution delivers personalized, actionable strategies, addressing complex, multifactorial challenges in a way that traditional, static methods cannot.

### SUMMARY

In one embodiment the invention provides a method for improving reproductive outcomes in a woman in need thereof, the method comprising:
delivering a therapeutic plan to the woman via a personal electronic device, wherein the therapeutic plan comprises therapeutic interventions selected from: at least one physical activity intervention, at least one psychological intervention, and at least one nutrition management intervention;
converting the therapeutic plan into a plurality of targeted interaction outputs for presentation to the woman; and
tracking the woman's responses and associated performance metrics throughout implementation of the therapeutic plan, and continuing until a predetermined condition is met.

In one embodiment the method further comprises administering at least one medicinal agent before, during, after, or any combination thereof, the implementation of at least one of the therapeutic interventions.

In one embodiment the at least one medicinal agent comprises a medicinal agent used in fertility treatment protocols or assisted reproductive technologies.

In one embodiment the fertility treatment protocols or assisted reproductive technology is selected from: in vitro fertilization protocols, ovulation induction, intrauterine insemination, controlled ovarian stimulation, luteal phase support, or combinations thereof.

In one embodiment the at least one medicinal agent comprises any of the following selected from: gonadotropins, gonadotropin-releasing hormone (GnRH) agonists, GnRH antagonists, human chorionic gonadotropin (hCG), progesterone, estrogen, letrozole, and clomiphene citrate, or any combination thereof.

In one embodiment the method further comprises administering at least one adjunct medicinal agent in combination with the at least one medicinal agent.

In one embodiment the method further comprises generating and delivering medication reminders to the woman.

In one embodiment the therapeutic plan is adapted to a phase of an IVF cycle selected from: suppression phase, ovarian stimulation phase, trigger phase, oocyte retrieval phase, fertilization phase, luteal phase support phase, embryo transfer phase, post-transfer phase, pregnancy testing phase, or any combination thereof.

In one embodiment the method further comprises delivering a conditioning stimulus before, during, after, or a combination thereof, the administering of the at least one medicinal agent, at least one of the therapeutic interventions, or a combination thereof.

In one embodiment the method further comprises obtaining a plurality of attributes associated with the woman to generate an initial profile for the woman, wherein the therapeutic plan is generated and/or customized based on the initial profile.

In one embodiment the plurality of attributes are selected from: age, ovarian reserve, hormone profile, reproductive history, prior response to ovarian stimulation, body mass index, genetic factors, comorbidities, medications, nutritional factors, exercise or lifestyle factors, mental wellbeing, inflammatory markers, insulin sensitivity, and patient-defined reproductive goals.

In one embodiment the method further comprises:
dynamically monitoring the woman's responses and associated performance metrics during implementation of the therapeutic plan;
generating a dynamic user profile based on the woman's responses and associated performance metrics and other parameters selected from: the initial profile, Electronic Medical Records (EMR) data, psychological attributes, cognitive attributes, pharmacological attributes, feedback of the woman, feedback of a medical practitioner, drug intake parameters, self-reported data, wearable device data, sensor data, and daily device usage patterns, or a combination thereof; and
recursively adapting the therapeutic plan based on the dynamic user profile.

In one embodiment the recursive adapting of the therapeutic plan comprises dynamically identifying and modifying intervention parameters to implement optimized therapeutic outcomes towards a predetermined therapeutic goal.

In one embodiment the intervention parameters comprise any of the following selected from: type, timing, dosage, intensity, sequence, duration, frequency, mode of delivery, level of guidance, feedback modality, language adaptation, complexity level, interface configuration, integration with external systems, gamification intensity, or a combination thereof, of one or more of the therapeutic interventions.

In one embodiment the recursive adapting is performed using a feedback control algorithm, Bayesian inference, machine learning models, AI-driven analytics, or a combination thereof.

In one embodiment the at least one physical activity intervention comprises at least one low-impact physical activity.

In one embodiment the method further comprises monitoring activity level during the at least one physical activity intervention using smartphone sensors, wearable technology, or a combination thereof, and providing personalized guidance based on real-time metrics selected from: step count, movement intensity, user-reported fatigue, distance, time, or a combination thereof.

In one embodiment the at least one psychological intervention is selected from: heart rate variability (HRV) biofeedback, guided imagery, psychoeducation, cognitive behavioral therapy, stress and anxiety management training, mindfulness-based interventions, body scanning training, sleep hygiene, fatigue training, pain psychology, pacing training, acceptance and commitment therapy (ACT), dialectical behavior therapy (DBT), attention training techniques, psychodynamic therapy, solution-focused brief therapy (SFBT), narrative therapy, gestalt therapy, behavioral activation therapy, adjustment therapy, grief therapy, motivational therapy, meaning-centered therapy, creative therapy, expressive therapy, logotherapy, progressive muscle relaxation, and breathing exercises, or a combination thereof.

In one embodiment the at least one nutrition management intervention comprises any of the following selected from:
delivering information on a set of dietary supplements and recording the woman's intake of each supplement at specific times relative to meals;
providing instructions for following one or more predetermined diet types, including instructions for substituting specified foods with designated healthier alternatives and preparing meals according to specific recipes;
delivering psychoeducational content that instructs the woman to follow a predetermined anti-inflammatory diet, including instructions for substituting specified foods with designated healthier alternatives and preparing meals according to specific recipes;
delivering dietary guidance configured to stabilize blood glucose levels and maintain insulin sensitivity, including instructions for timing and composition of food intake to maintain blood glucose within a predetermined healthy range;
providing meal logging and nutritional tracking functionality;
generating prompts at predetermined times after meal consumption that instruct the woman to perform the at least one of the therapeutic interventions; and
providing electronic notifications to the woman that prompt selection of designated healthy food options at scheduled eating times;
providing psychoeducational content and interactive guidance configured to reduce stress-related eating behaviors; or
any combination thereof.

In one embodiment each of the therapeutic interventions has a duration ranging between 10 s to 60 mins.

In one embodiment the predetermined condition is selected from: a predetermined number of iterations, a predetermined duration has elapsed, a predetermined number of therapeutic interventions have been completed, predetermined progress measurement, predetermined regression measurement, target threshold score achieved, transition to maintenance therapy, no further improvement is measured, achievement of a target follicle count or ovarian response, attainment of a target embryo quality or implantation potential, improvement in hormone levels relevant to reproductive function, improvement in endometrial receptivity markers or thickness, achieving a viable pregnancy, reaching a target threshold score in a fertility outcome metric or clinical scoring system, improvement in stress or psychological assessment scores, and reduction in inflammatory markers, or a combination thereof.

In one embodiment the targeted interaction outputs are selected from: visual, auditory, tactile, olfactory, thermal, or a combination thereof.

In one embodiment the improving reproductive outcomes comprises any of the following selected from: improving pregnancy outcomes, improving implantation rates, increasing the number of follicles, improving live birth outcomes, reducing miscarriage, increasing the likelihood that a woman initiates a subsequent treatment cycle following a failed cycle, improving egg quality, and improving embryo quality, or a combination thereof.

In one embodiment the method further comprises delivering gamification elements configured to engage the reward system and reinforce adherence to the therapeutic plan, adherence to the at least one medical agent regimen, exploration of additional fertility treatment options, or a combination thereof.

In one embodiment the invention provides a software application embodied on a non-transitory computer-readable medium, the application comprising instructions which, when executed by a processor, cause the processor to perform the methods of the invention.

In one embodiment the invention provides a digital therapeutic system for improving reproductive outcomes in a woman in need thereof, the system comprising:
a data-input module configured to onboard the woman and dynamically monitor and store data related to the woman throughout a therapeutic plan; and
a digital therapy delivery module comprising a processor configured to deliver to the woman a therapeutic plan comprising therapeutic interventions selected from: at least one physical activity intervention, at least one psychological intervention, and a nutrition management intervention;
wherein the digital therapy delivery module is further configured to convert the therapeutic plan into a plurality of targeted interaction outputs for presentation to the woman and track the woman's responses.

In one embodiment the data-input module is configured to obtain a plurality of attributes associated with the woman to generate an initial profile for the woman, and wherein the processor is further configured to generate and/or customize the therapeutic plan based on the initial profile.

In one embodiment the digital therapy delivery module comprises software executable on a personal electronic device.

In one embodiment the digital therapy delivery module is a smartphone.

In one embodiment the digital therapy delivery module comprises any of the following selected from: a personal electronic device, a graphical user interface, audio interface, haptic interface, olfactory interface, thermal interface, or a combination thereof.

In one embodiment the data-input module is configured to receive data from a plurality of data acquisition sources in real-time, wherein the data comprises one or more of: Electronic Medical Records (EMR) data, motion sensor data, position sensor data, environmental sensors data, audio sensors data, location sensors data, audio recording data, speech recognition data, gaze-tracking data, touch interaction data, physiological sensor data, optical sensor data, imaging sensors data, self-reported data, device usage pattern data, and camera data, or a combination thereof.

In one embodiment the processor is further configured to:
dynamically monitor any of the following factors of the woman selected from: responses and associated performance metrics, Electronic Medical Records (EMR) data, motor attributes, psychological attributes, cognitive attributes, pharmacological attributes, and drug intake parameters, self-reported data, wearable device data, device usage patterns, or a combination thereof, throughout the implementation of the therapeutic plan;
generate a dynamic user profile, and recursively adapt the therapeutic plan based on the dynamic user profile, performance metrics, and the woman's responses; or
a combination thereof.

In one embodiment the system further comprises at least one additional device operatively coupled to the digital therapy delivery module, selected from: health monitoring systems, medical devices, haptic devices, external speakers, headphones, virtual reality headsets, augmented reality glasses or devices, biofeedback sensors, wearable activity trackers, smartphones, tablets, smartwatches, personal computational devices, smart speakers, voice assistants, motion tracking sensors, virtual assistant systems, internet hubs, gait sensors, eye-tracking devices, voice recording devices, wearable devices, smell-releasing devices, and connected haptic devices, or combinations thereof.

In one embodiment the system further comprises a scheduling module configured to generate medication reminders according to each patient's dosage and timing.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A and 1B show representations of Center for Epidemiologic Studies Depression (CES-D) scores in subjects pre-and post-intervention i.e., decrease in depression scores with the intervention of the invention; for subjects with Subjective Cognitive Decline (SCD).

FIG. 2A shows intra-network connectivity in the brain. FIG. 2B shows a matrix showing significant connectivity differences between Default Mode Network (DMN) and Salience Network (SN) brain regions. FIG. 2C shows a graphical representation of changes in DMN-SN interconnectivity versus change CES-D scores. FIG. 2D shows a graphical representation of changes in DMN-SN interconnectivity versus change in Mental Health Continuum—Short Form (MHC-SF) score.

FIGS. 3A-3B show results for IL-18 levels decreased following the intervention. FIG. 3A shows IL-18 levels decreased by 25% post-intervention (p<0.05, d=−0.64, n=15). FIG. 3B shows the neuroimmune link by reduced IL-18, correlated with decreased rsFC between right amygdala and bilateral precuneus (posterior DMN). FIG. 3C illustrates a neuroimmune link: lower IL-18 levels were associated with reduced resting-state functional connectivity (rsFC) between the right amygdala and the bilateral precuneus, a key region of the posterior default mode network (DMN).

FIGS. 4A-4H show psychological questionnaire results. FIG. 4A shows percentage change from baseline for each group across psychological questionnaires. FIG. 4B shows a network of correlations among psychological measures, with stress, depression, and anxiety positively interrelated and overall mental health negatively associated with anxiety. FIGS. 4C-4H show questionnaire scores for various indicators: Perceived Stress Scale (PSS), Center for Epidemiologic Studies Depression Scale (CES-D), State-Trait Anxiety Inventory Short Form (STAIS-5), Mental Health Continuum-Short Form (MHC-SF), and Brief Resilient Coping Scale (BRCS).

FIGS. 5A-5B show immune mediator results. FIG. 5A shows change from baseline for each group across cytokines. FIG. 5B shows cumulative distributions of percentage decrease in cytokines. FIG. 5C shows a correlation network of cytokine interactions. Edge thickness represents the strength of correlation between cytokines, with red indicating positive correlations and blue indicating negative correlations, as shown by the color scale. Cytokines included are interleukin-12 (IL-12), interleukin-17 (IL-17), interferon-gamma (IFNγ), interleukin-23 (IL-23), tumor necrosis factor-alpha (TNFα), and monocyte chemoattractant protein-1 (MCP-1).

FIGS. 6A-6H generally show the association between brain plasticity, peripheral inflammation, and psychological state. FIGS. 6A-6B show the resting-state fMRI interaction effect: Test>Control, Post>Pre-intervention. FIGS. 6C-6E show the association between changes in insula connectivity and reductions in cytokine levels. FIGS. 6F-6H show the association between changes in insula connectivity and improvement in psychological scores.

FIGS. 7A-7C show the improvement of the Insomnia Severity Index (ISI) between groups.

FIG. 8A shows results for depression in Parkinson's disease (PD) patients, post intervention improvement in Beck's Depression Inventory II (BDI-II) scores. This improvement was correlated with brain connectivity changes within the limbic circuit. FIGS. 8B-8D show correlations between increased thalamic AV connectivity and improvement in depressive symptoms.

FIGS. 9A-9B show correlations between increased thalamic AV-mPFC connectivity and self-engagement with the software application of the invention for emotion regulation activities.

For simplicity and clarity of illustration, elements shown in the figures are not necessarily drawn to scale, and the dimensions of some elements may be exaggerated relative to other elements. In addition, reference numerals may be repeated among the figures to indicate corresponding or analogous elements.

### DETAILED DESCRIPTION

In various embodiments, the methods of the invention involve therapeutic interventions that are performed as standalone treatments without the use of any medicinal agents. Although the optional addition of medicinal agents can enhance the overall efficacy of the intervention, the core method remains one that does not require pharmaceutical intake. A description of embodiments involving the administration of medicinal agents is set forth in subsequent sections. In some embodiments, the therapeutic plan is delivered as a standalone digital intervention. In other embodiments, the therapeutic plan is delivered as part of a hybrid protocol that coordinates and/or synchronizes the digital interventions with administration of one or more medicinal agents and/or phases of a fertility treatment protocol.

In one embodiment the invention provides a method for improving reproductive outcomes in a woman in need thereof, the method comprising:
delivering a therapeutic plan to the woman via a personal electronic device, wherein the therapeutic plan comprises therapeutic interventions selected from: at least one physical activity intervention, at least one psychological intervention, and at least one nutrition management intervention;
converting the therapeutic plan into a plurality of targeted interaction outputs for presentation to the woman; and
tracking the woman's responses and associated performance metrics throughout implementation of the therapeutic plan, and continuing until a predetermined condition is met.

As used herein, the term “improving reproductive outcomes” refers to any enhancement in one or more parameters associated with fertility or assisted reproductive success. Such improvements include, but are not limited to: improving pregnancy outcomes, increasing implantation rates, increasing the number of follicles, improving live birth outcomes, reducing miscarriage, increasing the likelihood that a woman initiates a subsequent treatment cycle following a failed cycle, improving egg quality, improving embryo quality, or any combination thereof.

As used herein, the term “in need thereof” refers to a woman who may benefit from an intervention to enhance fertility or improve reproductive outcomes, whether she is currently pregnant (including early pregnancy following conception and/or embryo transfer) or not yet pregnant (including women preparing for or undergoing fertility evaluation and/or treatment, including assisted reproductive interventions (e.g., IVF)). Examples include, but are not limited to, women experiencing infertility or subfertility, undergoing fertility treatment protocols (including assisted reproductive technologies), recurrent implantation failure, poor egg or embryo quality, hormonal or endometrial dysregulation, advanced maternal age, previous failed IVF or other assisted reproductive cycles, or any other condition or circumstance in which improving reproductive outcomes would be desirable.

As used herein, “therapeutic plan” refers to a structured set of therapeutic interventions, strategies, or activities. The therapeutic plan may be personalized based on individual attributes, clinical data, behavioral responses, or performance metrics, and may include physical interventions, psychological interventions, nutritional interventions, or combinations thereof. It may be delivered digitally, adaptively modified over time, and presented through targeted interaction outputs.

As described herein, the “therapeutic interventions” refer to any number of therapeutic interventions. In one embodiment the therapeutic interventions comprise at least one physical intervention and at least one psychological intervention, either carried out together, or sequentially. Thus, the terms “therapeutic interventions” and “plurality of therapeutic interventions” can be understood interchangeably when referring to multiple therapeutic interventions.

For purposes of this disclosure, the terms “digital therapy” and “digital therapeutics” (or the likes) are used interchangeably with the term “therapeutic plan,” as each refers to a set of interventions delivered at least partially through a personal electronic device (smartphone).

As used herein “psychological” interventions are generally directed towards interventions that affect psychological and/or behavioral aspects of an individual. As such, psychological interventions are generally related to behavioral, social and psychological tasks, as will be described. As used herein “physical” interventions are refer to any intervention that involves physical action of the woman for a therapeutic purpose. Examples of physical interventions generally include physical exercise, such as low-impact or moderate intensity activities. As used herein, “nutrition management interventions” refer to any intervention that involves the management, modification, or optimization of a woman's dietary intake. Examples of such interventions include, but are not limited to, tailored meal planning, caloric or macronutrient adjustment, vitamin or mineral supplementation, dietary counseling, or other approaches aimed at improving reproductive, hormonal, or general health outcomes.

As used herein, and in one embodiment, “converting the therapeutic plan into a plurality of targeted interaction outputs for presentation to the woman” refers to the transformation of selected therapeutic interventions into user-perceivable stimuli and interactive elements that can be delivered through a digital interface. For example, as a software application on a smartphone. This process involves mapping each intervention type to appropriate output formats based on its functional goals, the capabilities of the user interface, and the woman's preferences or accessibility needs.

For example, physical interventions may be rendered as animated exercise demonstrations, touch-sensitive targets for coordination tasks, audio prompts for vocal exercises, or haptic feedback for rhythm training. Psychological interventions may be presented as video content for guided imagery, audio narration for mindfulness, or interactive questionnaires for cognitive behavioral therapy. The conversion process ensures that each therapeutic element is delivered in a format that is engaging, accessible, and aligned with the intended therapeutic outcome. In certain embodiments, determining the therapeutic plan may encompass any suitable mode of obtaining or defining the therapeutic plan for the individual. For example, the therapeutic plan may be acquired from an existing clinical protocol, guideline, standardized care pathway, or previously established therapy regimen. In other embodiments, the therapeutic plan may be generated de novo based on patient-specific characteristics, assessments, goals, or clinician input. In one embodiments, the therapeutic plan may be adapted or modified from a pre-existing plan, such as by revising one or more therapeutic elements, dosing schedules, behavioral interventions, or support activities in accordance with patient needs or observed responses. Any combination of acquiring, generating, and adapting a therapeutic plan is also contemplated, and the plan may continue to evolve dynamically over time in response to ongoing monitoring or feedback.

In one embodiment the invention provides a method for improving reproductive outcomes in a woman in need thereof, the method comprising:
delivering a therapeutic plan to the woman via a personal electronic device, wherein the therapeutic plan comprises therapeutic interventions selected from: at least one physical activity intervention, at least one psychological intervention, and at least one nutrition management intervention, or any combination thereof;
converting the therapeutic plan into a plurality of targeted interaction outputs for presentation to the woman; and
tracking the woman's responses and associated performance metrics throughout implementation of the therapeutic plan, and continuing until a predetermined condition is met.

In one embodiment the therapeutic plan comprises therapeutic interventions selected from two of: at least one physical activity intervention, at least one psychological intervention, and at least one nutrition management intervention.

In one embodiment the method further comprises obtaining a plurality of attributes associated with the woman to generate an initial profile for the woman, wherein the therapeutic plan is generated and/or customized based on the initial profile.

In one embodiment the plurality of attributes are selected from: age, ovarian reserve, hormone profile, reproductive history, prior response to ovarian stimulation, body mass index, genetic factors, comorbidities, medications, nutritional factors, exercise or lifestyle factors, mental wellbeing, inflammatory markers, insulin sensitivity, and patient-defined reproductive goals.

As used herein, the term “performance metric” refers to measurable indicators that reflect the woman's engagement, adherence, and progress in implementing the therapeutic plan. These performance metrics provide objective or subjective data points that enables the system to monitor effectiveness, personalize interventions, and determine when a predetermined condition (e.g., completion of a program or achievement of a goal) has been met.

In one embodiment the performance metrics include physical activity indicators, psychological indicators, nutrition-related indicators, engagement metrics, and outcome-oriented metrics, or any combination thereof.

Examples of physical activity indicators include, but not limited to: the number of completed exercise sessions, workout duration and intensity, step count, and heart rate variability. Examples of psychological indicators include, but are not limited to: stress level scores, mood tracking entries, completion of mindfulness exercises, and sleep quality ratings. Examples of nutrition-related indicators include, but are not limited to: daily caloric intake, macronutrient balance, meal logging frequency, and hydration tracking. Examples of engagement metrics include, but are not limited to: frequency of app interactions, response time to prompts, and completion rates of assigned tasks. Examples of outcome-oriented metrics include, but are not limited to: changes in body weight or BMI, improvements in hormonal profiles, and cycle regularity or ovulation tracking. Collectively, these performance metrics enable for adaptive, data-driven guidance throughout the therapeutic process.

In one embodiment the method further comprises:
dynamically monitoring the woman's responses and associated performance metrics during implementation of the therapeutic plan;
generating a dynamic user profile based on the woman's responses and associated performance metrics and other parameters selected from: the initial profile, Electronic Medical Records (EMR) data, psychological attributes, cognitive attributes, pharmacological attributes, feedback of the woman, feedback of a medical practitioner, drug intake parameters, self-reported data, wearable device data, sensor data, and daily device usage patterns, or a combination thereof; and
recursively adapting the therapeutic plan based on the dynamic user profile.

As used “recursively adapting” refers to an iterative and ongoing process of modifying the therapeutic plan based on updated information, including changes to the dynamic user profile, performance metrics, the woman's responses, and the likes.

In one embodiment, the recursive adapting of the therapeutic plan comprises dynamically identifying and modifying intervention parameters to optimize reproductive outcomes towards a predetermined therapeutic goal. In one embodiment, the predetermined therapeutic goal is aligned with the desired reproductive outcome. Examples of predetermined therapeutic goals include, but are not limited to: improving ovarian response, enhancing egg quality, increasing endometrial receptivity, regulating hormone levels, optimizing timing of ovulation or embryo transfer, increasing implantation rates, reducing risk of miscarriage, improving embryo development, supporting healthy pregnancy progression, enhancing psychological readiness and stress management, maintaining overall health and nutritional status, and achieving a target clinical outcome on a standardized fertility assessment or reproductive quality-of-life measure, etc.

In one embodiment the recursive adapting of the digitized therapeutic plan comprises dynamically identifying and modifying the therapeutic intervention parameters to implement optimized therapeutic outcomes towards a predetermined therapeutic goal.

In one embodiment the intervention parameters comprise any of the following selected from: type, timing, dosage, intensity, sequence, duration, frequency, mode of delivery, level of guidance, feedback modality, language adaptation, complexity level, interface configuration, integration with external systems, gamification intensity, or a combination thereof, of one or more of the therapeutic interventions.

Examples of ‘type’ include, but are not limited to: type of therapeutic intervention, type of medication, type of physical exercise, type of psychological intervention, type of nutritional intervention. Examples of ‘timing’ include, but are not limited to: time of day of intervention, timing relative to symptom onset, timing relative to medication administration, timing relative to patient-reported symptoms. Examples of ‘dosage’ include, but are not limited to: amount of medication, number of repetitions in an exercise session, number of tasks per session. Examples of ‘intensity’ include, but are not limited to: resistance level in a physical exercise, level of psychological challenge in a task, vibration amplitude in haptic feedback, degree of stretching or mobilization. Examples of ‘sequence’ include, but are not limited to: order of exercises, order of administered medications, and sequence of multi-modal interventions. Examples of ‘duration’ include, but are not limited to: length of each therapy session, duration of medication course, duration of continuous monitoring, duration of a feedback session. Examples of ‘frequency’ include, but are not limited to: number of sessions per week, frequency of medication administration, frequency of symptom reporting, frequency of device notifications. Examples of ‘mode of delivery’ include, but are not limited to: in-person, remote/digital delivery, virtual reality-based delivery, augmented reality-guided delivery. Examples of ‘level of guidance’ include, but are not limited to: fully guided intervention, self-directed intervention, therapist-assisted intervention, automated adaptive guidance. Examples of ‘feedback modality’ include, but are not limited to: visual feedback, auditory feedback, haptic feedback, real-time performance analytics. Examples of ‘language adaptation’ include, but are not limited to: different spoken languages, different written language formats, culturally adapted instructions, simplified versus technical terminology. Examples of ‘complexity level’ include, but are not limited to: simplified instructions, advanced task difficulty, multi-step cognitive tasks, multi-joint exercise coordination. Examples of ‘interface configuration’ include, but are not limited to: touchscreen interface, gesture-controlled interface, voice-controlled interface, multi-sensor integrated interface. Examples of ‘integration with external systems’ include, but are not limited to: wearable activity tracker, electronic health record system, home monitoring devices, cloud-based analytics platforms.

In one embodiment, the at least one physical activity intervention comprises at least one low-impact physical activity. In one embodiment, the therapeutic plan may include physical activity interventions delivered through a digital platform. The platform may include a “walk with me” feature for guided walking. This feature can optionally be accompanied by audio content such as podcasts or educational material related to sleep, wellbeing, stress reduction, and empowerment. The platform may also provide low-impact exercise routines. Examples of low-impact exercise routines include, but are not limited to: dance, light aerobic exercises, yoga adapted to avoid poses contraindicated during certain phases of treatment, Pilates, and Tai Chi. Guidance, instruction, motivation, and feedback may be provided to support adherence and engagement with the physical activity regimen. Other examples of low-impact exercises include walking in place, treadmill walking, elliptical machine workouts, swimming or water aerobics, resistance band exercises, bodyweight exercises such as gentle squats and seated leg lifts, stretching or mobility routines, balance exercises such as standing on one leg or stability ball exercises, core-strengthening exercises such as gentle Pilates or mat-based routines, and light cycling or stationary bike exercises.

In one embodiment the method further comprises monitoring activity level during the at least one physical activity intervention using smartphone sensors, wearable technology, or a combination thereof, and providing personalized guidance based on real-time metrics selected from: step count, movement intensity, user-reported fatigue, distance, time, or a combination thereof.

In one embodiment the at least one psychological intervention is selected from: heart rate variability (HRV) biofeedback, guided imagery, psychoeducation, cognitive behavioral therapy, stress and anxiety management training, mindfulness-based interventions, body scanning training, sleep hygiene, fatigue training, pain psychology, pacing training, acceptance and commitment therapy (ACT), dialectical behavior therapy (DBT), attention training techniques, psychodynamic therapy, solution-focused brief therapy (SFBT), narrative therapy, gestalt therapy, behavioral activation therapy, adjustment therapy, grief therapy, motivational therapy, meaning-centered therapy, creative therapy, expressive therapy, logotherapy, progressive muscle relaxation, and breathing exercises, or a combination thereof.

In one embodiment the at least one nutrition management intervention comprises any of the following selected from:
delivering information on a set of dietary supplements and recording user intake of each supplement at specific times relative to meals;
providing instructions for following one or more predetermined diet types, including instructions for substituting specified foods with designated healthier alternatives and preparing meals according to specific recipes;
delivering psychoeducational content that instructs the woman to follow a predetermined anti-inflammatory diet, including instructions for substituting specified foods with designated healthier alternatives and preparing meals according to specific recipes;
delivering dietary guidance configured to stabilize blood glucose levels and maintain insulin sensitivity, including instructions for timing and composition of food intake to maintain blood glucose within a predetermined healthy range;
providing meal logging and nutritional tracking functionality;
generating prompts at predetermined times after meal consumption that instruct the woman to perform the at least one of the therapeutic interventions; and
providing electronic notifications to the user that prompt selection of designated healthy food options at scheduled eating times;
providing psychoeducational content and interactive guidance configured to reduce stress-related eating behaviors; or
any combination thereof.

Any of the listed nutrition management interventions may be implemented alone or in any combination. In one embodiment the nutrition management intervention comprises transitioning to a Mediterranean diet.

In certain embodiments, the nutrition management intervention further includes strategies to address stress-related eating behaviors. These strategies may comprise delivering psychoeducational content that explains the impact of stress eating on inflammation and blood glucose regulation, providing interactive exercises to enhance awareness of hunger cues and emotional triggers, and generating prompts or notifications that encourage mindful consumption of food. Such interventions aim to reduce the likelihood of stress-induced dietary choices and support adherence to nutritional goals within the therapeutic plan.

In one embodiment each of the therapeutic interventions has a duration ranging between 10 s to 60 mins. In one embodiment each of the therapeutic interventions has a duration ranging between 10 s to 30 mins. In one embodiment each of the therapeutic interventions has a duration ranging between 10 s to 10 mins. In one embodiment each of the therapeutic interventions has a duration ranging between 10 s to 50 mins. In one embodiment each of the therapeutic interventions has a duration ranging between 10 s to 2 mins. In one embodiment each of the therapeutic interventions has a duration ranging between 1 min to 30 mins. In one embodiment each of the therapeutic interventions has a duration ranging between 2 mins to 30 mins. In one embodiment each of the therapeutic interventions has a duration ranging between 5 mins to 30 mins. In one embodiment each of the therapeutic interventions has a duration ranging between 0.5 min to 10 mins.

In one embodiment the predetermined condition is selected from: a predetermined number of iterations, a predetermined duration has elapsed, a predetermined number of therapeutic interventions have been completed, predetermined progress measurement, predetermined regression measurement, target threshold score achieved, transition to maintenance therapy, no further improvement is measured, achievement of a target follicle count or ovarian response, attainment of a target embryo quality or implantation potential, improvement in hormone levels relevant to reproductive function, improvement in endometrial receptivity markers or thickness, achieving a viable pregnancy, reaching a target threshold score in a fertility outcome metric or clinical scoring system, improvement in stress or psychological assessment scores, and reduction in inflammatory markers, or a combination thereof

In one embodiment the targeted interaction outputs are selected from: visual, auditory, tactile, olfactory, thermal, or a combination thereof.

In one embodiment the method further comprises delivering gamification elements configured to engage the reward system and reinforce adherence to the therapeutic plan, adherence to the at least one medical agent regimen, exploration of additional fertility treatment options, or a combination thereof. The gamification elements act as a complementary layer that amplifies the impact of treatment by reinforcing positive behaviors.

### Methods of the Invention Comprising Administration of Medicinal Agents

Various pharmacological approaches in IVF span several stages of the treatment cycle, each relying on precise hormonal manipulation. During ovarian stimulation, patients typically receive gonadotropins, such as recombinant follicle-stimulating hormone (rFSH) or human menopausal gonadotropin (hMG) and recombinant luteinizing hormone (rLH), either alone or in combination, in order to induce the development of multiple follicles. To prevent premature ovulation, clinicians add either GnRH agonists or GnRH antagonists. Agonists like leuprolide work by suppressing the body's natural LH surge over time, while antagonists such as cetrorelix and ganirelix provide an immediate suppression of LH release. In one embodiment the medicinal agent is Pergoveris.

When follicles reach maturity, the cycle moves to the final oocyte maturation trigger, using either human chorionic gonadotropin (hCG) or, depending on the protocol, a GnRH agonist to mimic the physiological LH surge necessary for oocyte maturation prior to retrieval.

Following egg retrieval, luteal phase support becomes essential to prepare and maintain the endometrium for implantation. This is most commonly achieved with progesterone, administered via intramuscular injections, vaginal gels, or suppositories. In some protocols, hCG is added alongside progesterone to further reinforce luteal function.

In various embodiments, a range of supplements and adjunct medications are also used to optimize implantation or address individualized clinical needs. Estrogen can be prescribed to improve endometrial receptivity, while low-dose aspirin or anticoagulants are used in cases where clotting disorders or immune factors pose a risk to implantation. In various embodiments, antibiotics are given prophylactically around the time of egg retrieval or before embryo transfer. Additionally, some patients receive low-dose corticosteroids to reduce inflammation or mitigate immune-related barriers.

Other medications frequently support cycle preparation. Oral contraceptives are often used at the outset to synchronize follicular development and to help schedule treatment. In long protocols, GnRH agonists are again employed for extended ovarian suppression to stabilize endogenous hormone levels before stimulation begins.

In various embodiments, these medications are administered in carefully timed regimens tailored to the patient's medical history, ovarian reserve, and response to treatment.

The success of IVF is significantly influenced by patient-related factors, such as age, ovarian reserve, and pre-existing health conditions. Additionally, lifestyle factors like smoking, excessive alcohol consumption, obesity, poor nutrition, and high stress levels have been shown to reduce fertility and compromise IVF success rates. Addressing these factors through early intervention and lifestyle modification can help optimize treatment outcomes.

Stress is a well-recognized factor that can negatively impact fertility, creating a complex and often cyclical relationship. Chronic stress affects the hypothalamic-pituitary-gonadal (HPG) axis, which plays a central role in regulating reproductive hormones. Elevated levels of stress hormones, such as cortisol, can disrupt ovulation and menstrual cycles, thereby reducing the likelihood of conception. In addition, the emotional strain of infertility itself can exacerbate stress, creating a feedback loop that further impairs reproductive outcomes.

Stress can negatively influence IVF outcomes, affecting egg retrieval, embryo transfer, and the likelihood of achieving pregnancy.

Additionally, prolonged stress can dysregulate the immune system by increasing pro-inflammatory cytokines and altering immune tolerance, both of which are crucial for successful conception and implantation. Abnormal production of pro-and anti-inflammatory cytokines, their receptors and other immune factors such as natural-killer (NK) cells, results in embryo implantation failure and pregnancy loss. Dysregulation of the interleukin network can jeopardize implantation and contribute to recurrent implantation failure (RIF)

Physical activity plays a crucial role in fertility, particularly through its impact on metabolic health and blood sugar regulation. Regular, moderate exercise improves insulin sensitivity, helping to stabilize blood sugar levels and reduce the risk of insulin resistance, a condition linked to ovulatory disorders such as polycystic ovary syndrome (PCOS). Balanced blood sugar levels are vital for maintaining hormonal harmony, as insulin resistance can disrupt the HPG axis, impairing ovulation and egg quality. Even in lean women without PCOS, insulin resistance was associated with a poor percentage of mature eggs and poor embryo quality. Excessive physical activity, however, can lead to hormonal imbalances and negatively affect reproductive outcomes. Therefore, maintaining a moderate level of exercise, combined with a balanced diet to regulate blood glucose, can optimize metabolic and hormonal health, enhancing fertility and improving the success of natural conception and fertility treatments.

Fertility outcomes are also impacted by kisspeptin, a key regulator of the reproductive axis, which is linked to stress, the immune system, and lifestyle factors.

Kisspeptins are a family of peptides, encoded by the Kiss1 gene, that operate via the surface receptor, Gpr54 (also called Kiss1r), to regulate virtually all aspects of reproduction in both sexes. The primary site of action of kisspeptins is the hypothalamus, where Kiss1 neurons engage in the precise control of the pulsatile release of GnRH to modulate gonadotropin secretion and, thereby, ovarian function. Nonetheless, additional sites of action of kisspeptins within the hypothalamic-pituitary-ovarian (HPO) axis, including the pituitary and the ovary, have been proposed.

Chronic stress elevates cortisol levels, suppressing kisspeptin activity and disrupting hormonal balance. Furthermore, kisspeptin neurons plays a bridging role as intermediary between the hypothalamic-pituitary-adrenal (HPA) axis (stress response system) and the HPG axis (reproductive regulatory system). Kisspeptin is also plays a role of an integrator of reproductive and immune functions during pregnancy. Immune stress reduces kisspeptin receptor mRNA, and kisspeptin has been implicated in the regulation of specific cytokines during pregnancy.

Diet and exercise also affect kisspeptin function-nutritional deficiencies, excessive exercise and obesity can alter kisspeptin signaling through metabolic disruptions.

The methods of the invention comprise the integration of combinations of six complimentary modes of action within a single treatment protocol: (1) reducing stress and inflammation, (2) physical activity, (3) nutrition management, (4) rebalancing emotional, cognitive, and hormonal networks for fertility, (5) enhancing brain plasticity, and (6) triggering the reward system. As will be described, these modes of action can be implemented individually or in various combinations within a single treatment protocol, depending on the specific needs and conditions of the individual.

The different modes are designed to work in concert, where each mode reinforces and enhances the efficacy of the others. A treatment protocol based on this method is thus predicated on a careful selection, design, and ordering of interventions, designed to deliver a therapeutic impact which is greater than the sum of its parts. The treatment parameters are subject to algorithmic personalization and optimization based on multiple data points, including individual response patterns. While the ideal treatment protocol involves the incorporation of all modes of action, in certain embodiments of this invention, the protocols of treatment include only a subset (one or more than one) of these modes, and in other treatment protocols, certain modes are emphasized more than others at different points in the therapeutic process. Some of the digital interventions integrated in the protocol are designed primarily to deliver one of the principal modes of action described above and address one of the factors contributing to infertility. However, in some cases, a single intervention may span more than one mode of action.

In one embodiment the method further comprises administering at least one medicinal agent before, during, after, or any combination thereof, the implementation of at least one of the therapeutic interventions.

As used herein, “administration” refers to the delivery, provision, or introduction of a medicinal agent to a woman in need thereof for the purpose of achieving a therapeutic effect. Examples of administration include, but are not limited to: oral, parenteral, subcutaneous, intramuscular, intravenous, vaginal, transdermal, or mucosal routes.

In one embodiment the at least one medicinal agent comprises a medicinal agent used in fertility treatment protocols or assisted reproductive technologies.

As used herein, “fertility treatment protocols” refer to any systematic regimen, plan, or sequence of interventions, procedures, or treatments administered to a woman for the purpose of enhancing, supporting, or regulating her fertility or reproductive function. As used herein, “assisted reproductive technologies” (ART) refer to any medical, laboratory, or procedural interventions performed in a woman as part of efforts to facilitate conception, support reproduction, or improve reproductive outcomes.

In one embodiment the fertility treatment protocols or assisted reproductive technology are selected from: in vitro fertilization protocols, ovulation induction, intrauterine insemination, controlled ovarian stimulation, luteal phase support, or combinations thereof.

Examples of medicinal agents comprises any of the following selected from: gonadotropins, gonadotropin-releasing hormone (GnRH) agonists, GnRH antagonists, human chorionic gonadotropin (hCG), progesterone, estrogen, letrozole, and clomiphene citrate, or any combination thereof.

Examples of gonadotropins include, but are not limited to: recombinant follicle-stimulating hormone (rFSH), human menopausal gonadotropin (hMG), and recombinant luteinizing hormone (rLH). Examples of gonadotropin-releasing hormone (GnRH) agonists include, but are not limited to: leuprolide acetate (leuprolide), nafarelin acetate (nafarelin), and triptorelin acetate (triptorelin). Examples of human chorionic gonadotropin (hCG) include, but are not limited to: urinary-derived chorionic gonadotropin for injection (e.g., Pregnyl, Novarel, Profasi, Choragon) and recombinant choriogonadotropin alfa (e.g., Ovidrel). Examples of gonadotropin-releasing hormone (GnRH) antagonists include, but are not limited to: cetrorelix acetate (cetrorelix) and ganirelix acetate (ganirelix). Examples of progesterone formulations include, but are not limited to: progesterone in oil for intramuscular injection, micronized progesterone capsules, vaginal progesterone gel (e.g., Crinone), vaginal progesterone inserts (e.g., Endometrin), and dydrogesterone. Examples of estrogen formulations include, but are not limited to: estradiol valerate, micronized estradiol, estradiol transdermal patches, and estradiol hemihydrate.

In one embodiment the method further comprises administering at least one adjunct medicinal agent in combination with the at least one medicinal agent.

Examples of adjunct medications include, but are not limited to: letrozole, clomiphene citrate, aspirin, anticoagulants, antibiotics, steroids, (oral) contraceptives, supplements. Examples of steroids (corticosteroids) include, but are not limited to: prednisone, methylprednisolone, hydrocortisone, dexamethasone, and betamethasone. In one embodiment the adjunct medicinal agent comprises biguanide. Examples of biguanides include, but are not limited to: Metformin. In one embodiment the adjunct medicinal agent comprises insulin-sensitizing drugs. Examples of insulin-sensitizing drugs include, but are not limited to: Thiazolidinediones, GLP-1 receptor agonists, DPP-4 inhibitors, etc.

Examples of oral contraceptives include, but are not limited to: combined estrogen-progestin pills, progestin-only pills, and combination pills containing ethinyl estradiol and levonorgestrel. Examples of antibiotics include, but are not limited to: doxycycline, azithromycin, amoxicillin, ceftriaxone, and clindamycin. Examples of anticoagulants include, but are not limited to: heparin, low-molecular-weight heparins (e.g., enoxaparin), warfarin, and fondaparinux. Examples of supplements include, but are not limited to, vitamins (e.g., folic acid, vitamin D), minerals (e.g., iron, zinc, selenium), and antioxidants. Examples of analgesics include, but are not limited to: acetaminophen (paracetamol), ibuprofen, codeine, or other pain-relieving agents suitable for use during fertility treatment, or any combination thereof.

In one embodiment the method further comprises generating and delivering medication reminders to the woman.

In one embodiment the therapeutic plan is adapted to a phase of an IVF cycle selected from: suppression phase, ovarian stimulation phase, trigger phase, oocyte retrieval phase, fertilization phase, luteal phase support phase, embryo transfer phase, post-transfer phase, pregnancy testing phase, or any combination thereof.

During the suppression phase, medications may be administered to suppress natural hormone production prior to ovarian stimulation. In various embodiments the therapeutic plan may provide guidance on preparing the body and mind for the upcoming IVF cycle, including stress reduction, sleep optimization, nutrition, and physical activity support

During the ovarian stimulation phase, injections of gonadotropins are administered to stimulate follicle growth. In various embodiments the therapeutic plan includes reminders for medication administration, monitoring of doses, and guidance for managing injection-related discomfort. Psychological support may be provided to reduce stress, improve sleep, and maintain motivation. Nutrition and physical activity guidance may also be provided to support overall health and prepare for egg retrieval.

During the trigger phase, the trigger shot is administered, typically approximately 36 hours before oocyte retrieval. In various embodiments the therapeutic plan includes guidance for timing and administration of the injection, as well as support to ensure adherence and reduce anxiety.

During the oocyte retrieval phase, eggs are retrieved at the scheduled time following the trigger shot. In various embodiments the therapeutic plan includes a predetermined protocol aimed at reducing stress, providing explanations of the procedure, offering practical guidance for dos and don'ts, and supporting the patient throughout the process.

During the fertilization phase, the retrieved eggs are fertilized in the laboratory. The therapeutic plan may continue to provide psychological support, maintain physical activity, and offer guidance to prepare the patient for the embryo transfer procedure.

During the luteal phase support phase, hormonal supplementation is administered following retrieval or transfer to support implantation. In various embodiments the therapeutic plan includes reminders for hormone administration, guidance for managing side effects, and continued psychological and lifestyle support.

During the embryo transfer phase, the embryo is transferred, typically 2-5 days after retrieval. In various embodiments the therapeutic plan includes specialized protocols for transfer day, guidance on hormone supplementation, stress reduction techniques, and practical instructions before, during, and after the procedure.

During the post-transfer phase, often referred to as the “two-week wait,” the patient experiences uncertainty and passivity while awaiting pregnancy testing. In various embodiments the therapeutic plan provides interventions to address stress, promote well-being, and offer reminders for appointments and self-care.

During the pregnancy testing phase, blood tests are performed to measure hCG levels and confirm pregnancy. In various embodiments the therapeutic plan includes reminders for tests, guidance on interpreting results, and psychological support tailored to the outcome of the testing.

In one embodiment the method further comprises delivering a conditioning stimulus before, during, after, or a combination thereof, the administering of the at least one medicinal agent, at least one of the therapeutic interventions, or a combination thereof. As used herein “conditioning” (or “conditioning stimulus”) refers to the process of associating a particular cue or stimulus with the administration of a drug or medication, or any other type of intervention (e.g., digitized therapeutic intervention), in order to promote positive effects or outcomes. In one embodiment the conditioning stimulus is delivered before, during, after, or a combination thereof, the administering of the at least one medicinal agent, at least one of the therapeutic interventions, or a combination thereof. When therapeutic interventions are delivered in conjunction with the administration of medicinal agents, the combination may produce a priming effect, wherein the therapeutic activity sensitizes or prepares the individual's physiological or cognitive systems to respond more effectively to the pharmacological treatment, thereby enhancing overall therapeutic efficacy. In certain embodiments, the conditioning stimulus is used to reinforce associative learning between a sensory cue and the therapeutic or pharmacological effect when drug administration is involved. Otherwise, the conditioning stimulus can also be used in combination with the standalone digitized therapeutic interventions themselves. For example, a distinct light pulse, vibration, or tone may be paired with drug administration or with the initiation of a digital intervention, enabling conditioned therapeutic responses and supporting neurobehavioral reinforcement even in the absence of the active stimulus. Non-limiting examples of sensory cues include auditory cues (e.g., tones, music, verbal prompts), visual cues (e.g., images, animations, and/or color-coded cycle indicators), and haptic cues (e.g., vibration patterns), delivered via the personal electronic device and/or associated peripherals. This associative pairing enhances expectancy, adherence, and long-term consolidation of treatment gains.

In certain embodiments, the digital therapeutic system further comprises AI-driven analytics designed to enhance personalization and treatment efficacy. These analytics may include AI-based identification of maladaptive thought patterns through analysis of user inputs and behavioral data, enabling early detection of cognitive barriers to adherence. Additionally, the system may employ AI-powered motivational interviewing techniques to deliver dynamic, context-sensitive prompts that encourage engagement and reinforce positive behavioral change. Furthermore, AI-driven algorithms can continuously adjust the therapeutic plan in real time based on performance metrics, user feedback, and predictive modeling, ensuring that interventions remain relevant and effective throughout the treatment process. Collectively, these AI-driven capabilities provide a highly adaptive and individualized approach to improving reproductive outcomes.

### Systems and Software Application of the Invention

In one embodiment the invention provides a software application embodied on a non-transitory computer-readable medium, the application comprising instructions which, when executed by a processor, cause the processor to perform the methods of the invention. As used herein “software application” refers to a set of computer-executable instructions, stored on a non-transitory computer-readable medium, configured to cause a computing device (e.g., a smartphone) to perform a sequence of operations corresponding to one or more steps of the methods of the invention. In one embodiment the invention provides a software application to execute the methods of the invention.

In one embodiment the instructions are executed at least partially on a mobile computing device e.g., a smartphone. In one embodiment the instructions are executed on a server or cloud computing system. In one embodiment the instructions are executed at least partially on a server or cloud computing system.

In one embodiment the invention provides a digital therapeutic system for improving reproductive outcomes in a woman in need thereof, the system comprising:
a data-input module configured to onboard the woman and dynamically monitor and store data related to the woman throughout a therapeutic plan; and
a digital therapy delivery module comprising a processor configured to deliver to the woman a therapeutic plan comprising therapeutic interventions selected from: at least one physical activity intervention, at least one psychological intervention, and a nutrition management intervention;
wherein the digital therapy delivery module is further configured to convert the therapeutic plan into a plurality of targeted interaction outputs for presentation to the woman and track the woman's responses.

As used herein, the “data-input module” refers to a software and/or hardware component configured to onboard the woman and continuously capture, monitor, and store data throughout the therapeutic process. This module may include interfaces for manual data entry by the woman (e.g., questionnaires, symptom logs, meal tracking), automated data collection from connected devices or sensors (e.g., wearable fitness trackers, smart scales), and integration with external health data sources. The data-input module ensures that relevant physical, physiological, behavioral, and engagement data are dynamically updated and securely stored for use in personalizing and adjusting the therapeutic plan.

As used herein the “digital therapy delivery module” refers to a processor-controlled component responsible for generating and delivering the therapeutic plan to the woman via a personal electronic device. The digital therapy delivery module converts the therapeutic plan into targeted interaction outputs, such as notifications, prompts, multimedia content, and interactive exercises, and presents them in a user-friendly format. In one embodiment the digital therapy delivery module tracks user responses and engagement with these outputs, enabling real-time monitoring and adaptive adjustments to the therapeutic plan. In one embodiment digital therapy delivery module incorporates AI-driven analytics to personalize interventions and optimize outcomes.

In one embodiment the data-input module is configured to obtain a plurality of attributes associated with the woman to generate an initial profile for the woman, and wherein the processor is further configured to generate and/or customize the therapeutic plan based on the initial profile.

In one embodiment the digital therapy delivery module comprises software executable on a personal electronic device. In one embodiment the digital therapy delivery module is a smartphone. In one embodiment the digital therapy delivery module comprises any of the following selected from: a personal electronic device, a graphical user interface, audio interface, haptic interface, olfactory interface, thermal interface, or a combination thereof.

As used herein, the term “personal electronic device” is understood to be any electronic device used by a user to carry out any part of the digital intervention. Thus, although it is often a ‘personal’ device, it is also understood to include devices that are not directly owned by the user, but those that are used/synced for the user to implement the digital intervention. The following are examples of personal electronic device, but are not limited to: smartphones, tablets, wearable device, smart TVs, computers, laptops, E-readers, gaming consoles, smartwatches, fitness trackers, portable media players, digital cameras, virtual reality (VR) headsets, augmented reality (AR) device, portable GPS devices, portable Bluetooth devices, portable digital assistant, smart glasses and audio device or any combinations thereof. In various embodiments the personal electronic device is a mobile computing device.

In one embodiment the data-input module is configured to receive data from a plurality of data acquisition sources in real-time, wherein the data comprises one or more of: Electronic Medical Records (EMR) data, motion sensor data, position sensor data, environmental sensors data, audio sensors data, location sensors data, audio recording data, speech recognition data, gaze-tracking data, touch interaction data, physiological sensor data, optical sensor data, imaging sensors data, self-reported data, device usage pattern data, and camera data, or a combination thereof. Examples of data acquisition sources includes, but are not limited to: electronic medical record (EMR) data, clinical practice guidelines, evidence-based protocols, patient input, expert consultation including medical professionals, allied health professionals, and user experience specialists, and machine learning analysis of aggregated patient outcome data to identify and optimize effective therapeutic intervention combinations. Electronic Medical Records (EMR) refers to digital medical records maintained by a healthcare provider for an individual patient. Comprised within that, the EMR data can include any of the following: patient demographics, medical history and prior diagnoses, current and past medications and prescriptions, documented allergies and contraindications, vital signs and other routine clinical measurements, laboratory test results, imaging reports (e.g., X-ray, CT, MRI), clinician notes, treatment plans and care instructions, and records of visits, appointments, and clinical encounters.

In one embodiment the processor is further configured to:
dynamically monitor any of the following factors of the woman selected from: responses and associated performance metrics, Electronic Medical Records (EMR) data, motor attributes, psychological attributes, cognitive attributes, pharmacological attributes, and drug intake parameters, self-reported data, wearable device data, device usage patterns, or a combination thereof, throughout the implementation of the therapeutic plan;
generate a dynamic user profile, and recursively adapt the therapeutic plan based on the dynamic user profile, performance metrics, and the woman's responses; or
a combination thereof.

In one embodiment the system further comprises at least one additional device operatively coupled to the digital therapy delivery module, selected from: health monitoring systems, medical devices, haptic devices, external speakers, headphones, virtual reality headsets, augmented reality glasses or devices, biofeedback sensors, wearable activity trackers, smartphones, tablets, smartwatches, personal computational devices, smart speakers, voice assistants, motion tracking sensors, virtual assistant systems, internet hubs, gait sensors, eye-tracking devices, voice recording devices, wearable devices, smell-releasing devices, and connected haptic devices, or combinations thereof.

In one embodiment the system further comprises a scheduling module configured to generate medication reminders according to each patient's dosage and timing.

### EXAMPLES

### Example 1

### Clinical Results for the Digital Interventions of the Invention

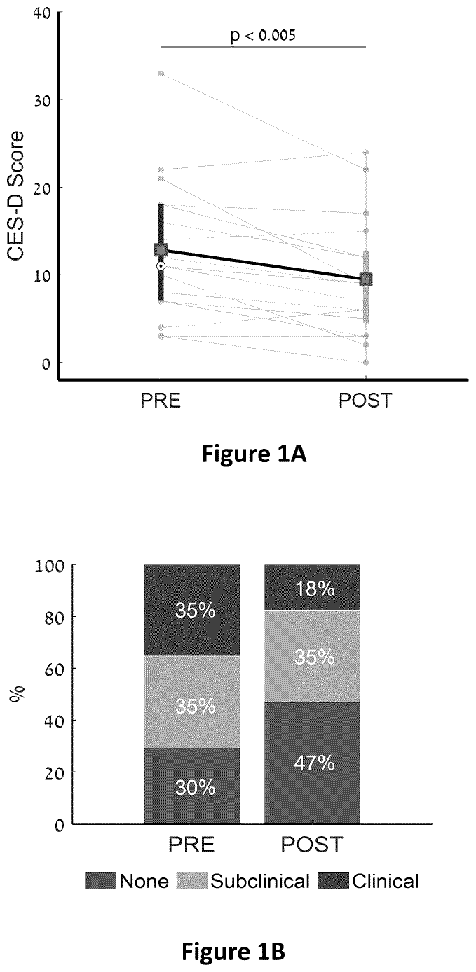

FIG. 1A shows significant reduction in depression scores post-intervention: CES-D scores dropped by 26.6%(p=0.004, d=−0.829). FIG. 1B shows 18% fewer participants met the depression cut-off; 17% more classified as “no depression.”

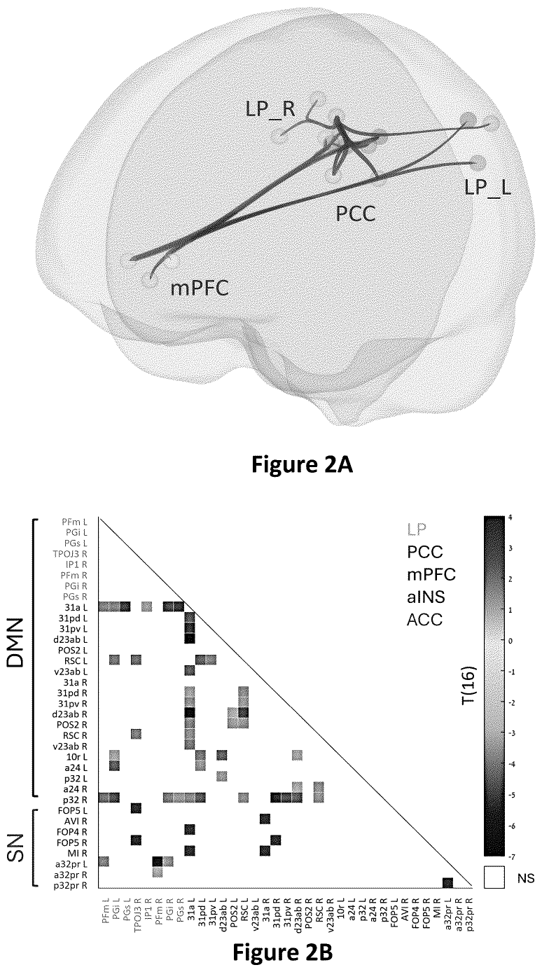

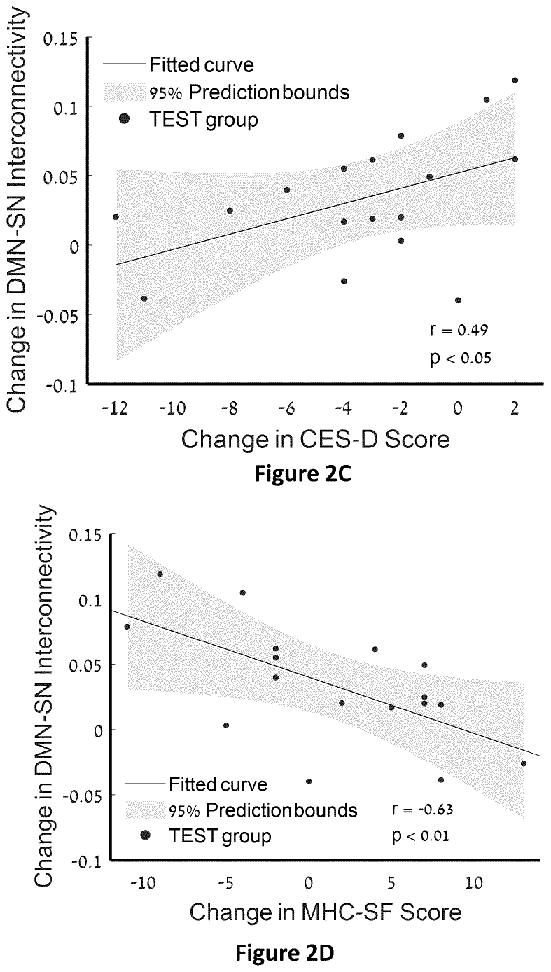

FIGS. 2A-2D show brain cognitivity analysis. Post-intervention changes in DMN: Significant alterations in intra-network connectivity, and clinical correlation: Greater depression score improvement was associated with increased negative DMN-SN rsFC. The DMN is a global brain network, which shows higher activity during rest, and is involved with self-referential processes, recalling memories and daydreaming. Increased rsFC within the DMN is associated with psychological processes and may reflect several clinical aspects of depression, such as rumination, repetitive negative thinking, and unforgiveness. In the present study, we demonstrated post-intervention reduction in the intrinsic connectivity within the DMN, specifically, a reduced connectivity between the PCC node and mPFC and LP nodes. It is noted that increased connectivity is demonstrated in late life depression, compared to non-depressed subjects, between the PCC and the anterior DMN, mPFC and sgACC. This reduced connectivity may explain the improvement in depression score, as the connectivity between the anterior and posterior DMN regions has been shown to reflect positive treatment effects.

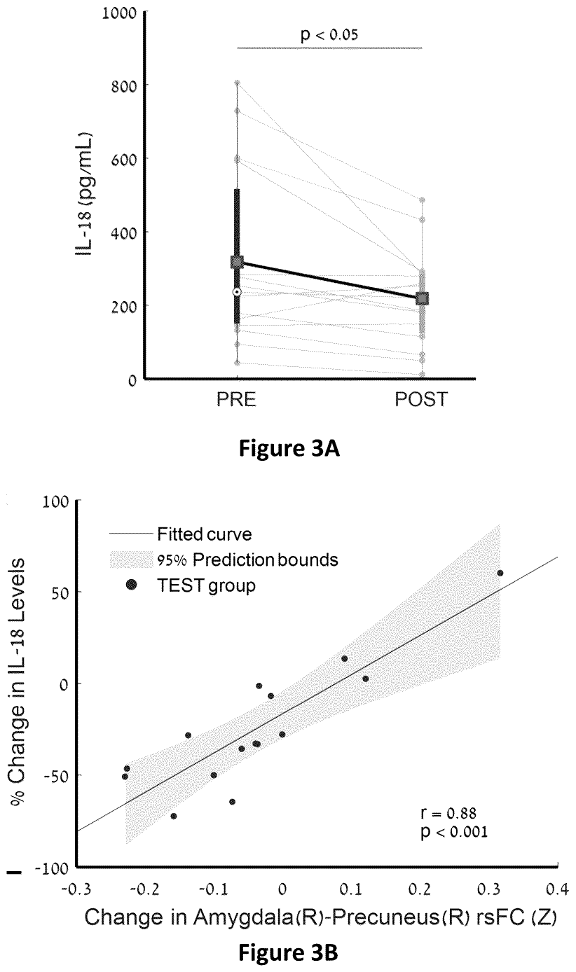

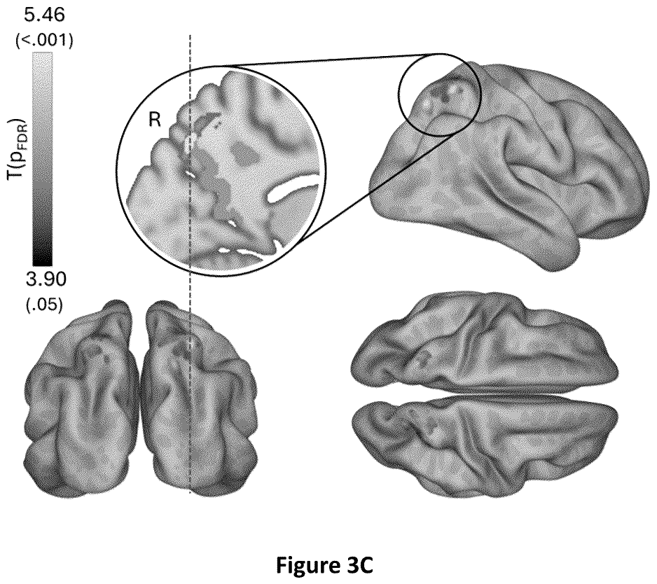

FIGS. 3A-3B show IL-18 levels decreased by 25% post-intervention (p<0.05, d=−0.64, n=15) and neuroimmune link: Reduced IL-18 was correlated with decreased rsFC between right amygdala and bilateral precuneus (posterior DMN). Thus the digital interventions of the invention resulted in reduced depressive symptoms and decreased inflammation marker, correlated with changes in brain connectivity.

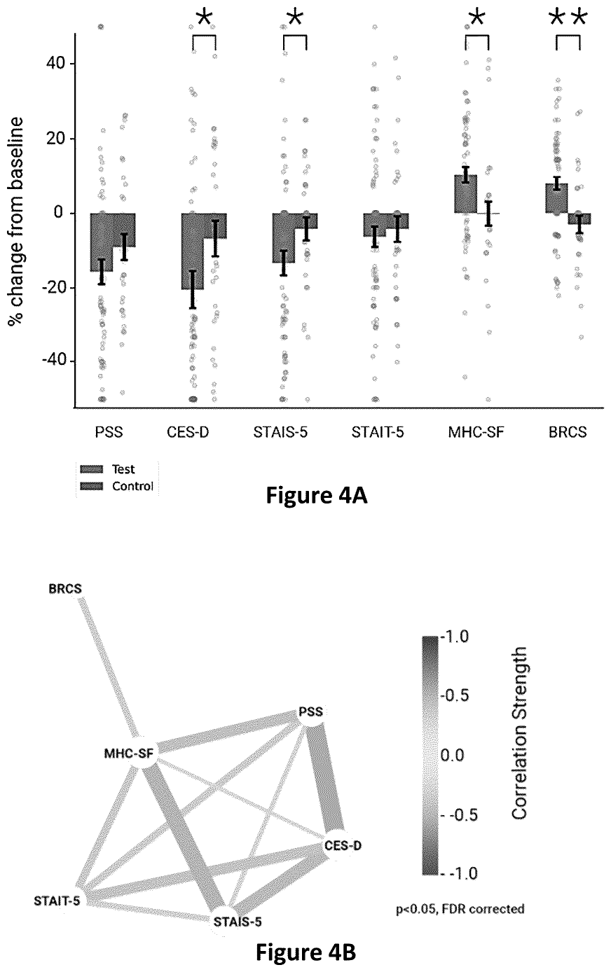

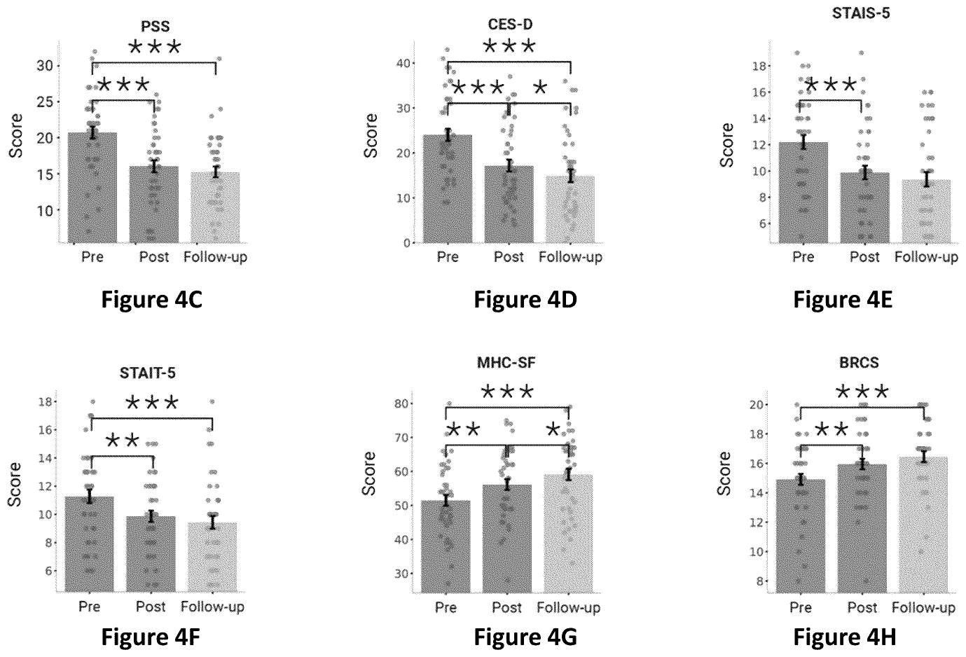

FIGS. 4A-4H show significant improvements observed in the intervention group across key psychological domains and that these positive outcomes were sustained or even enhanced following a 3-week low-intensity follow-up in some domains.

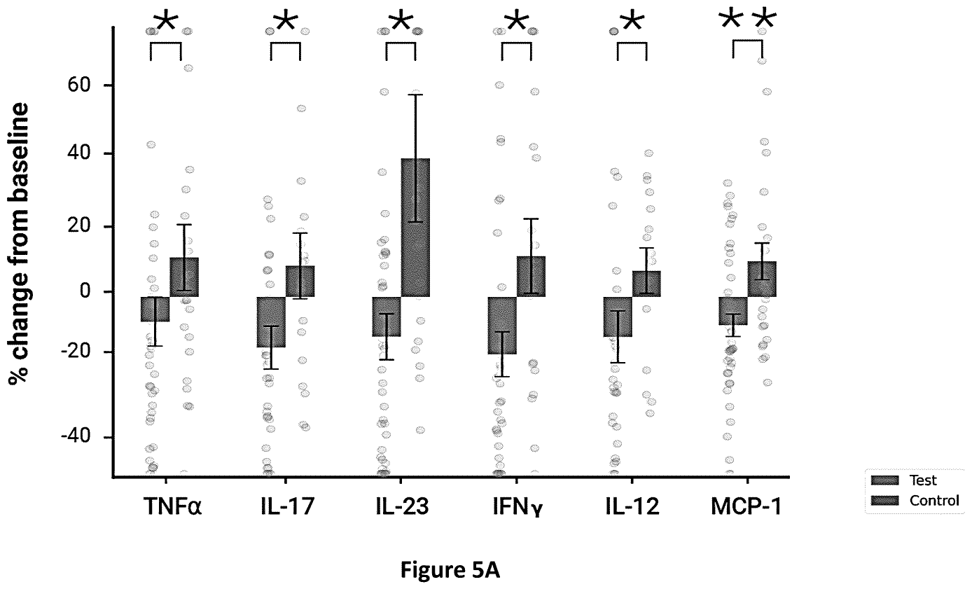

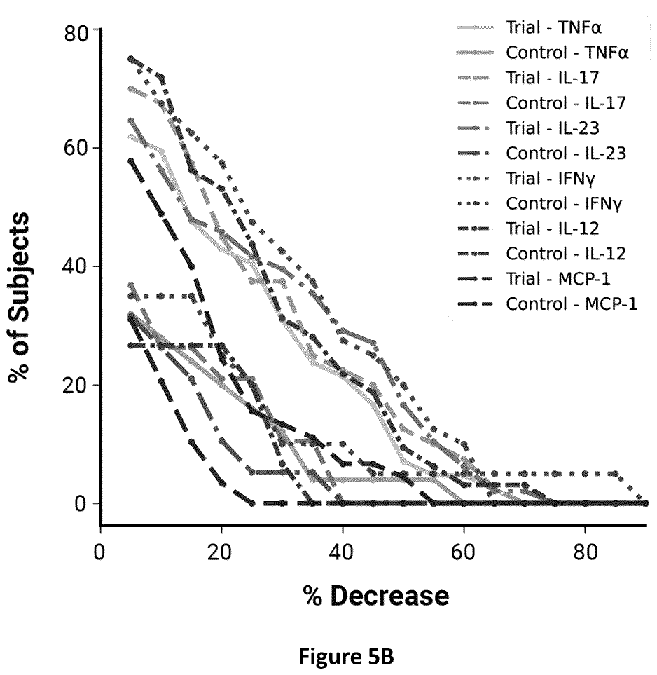

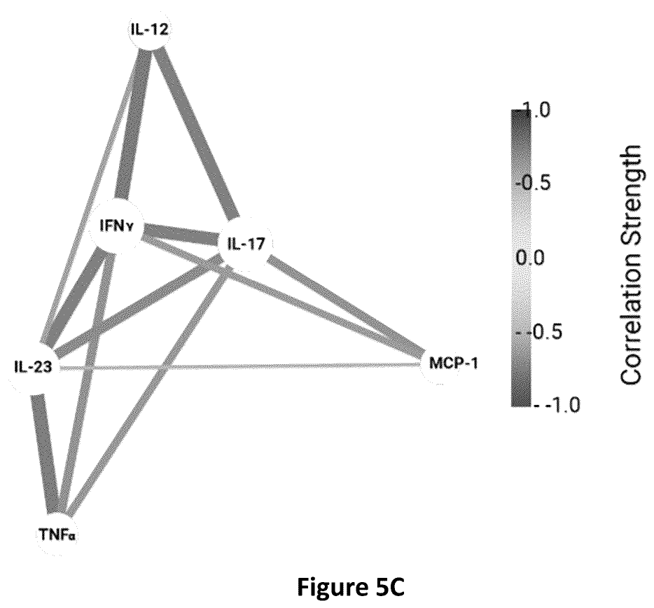

FIGS. 5A-5B show immune mediator results. Significant group-by-time effects were observed for multiple cytokines: TNF-α, IL-17, IL-23, MCP-1, IFN-γ, and IL-12. Furthermore, greater reductions (>5%) in these cytokines were more common in the intervention group, with significant differences vs. control across all markers.

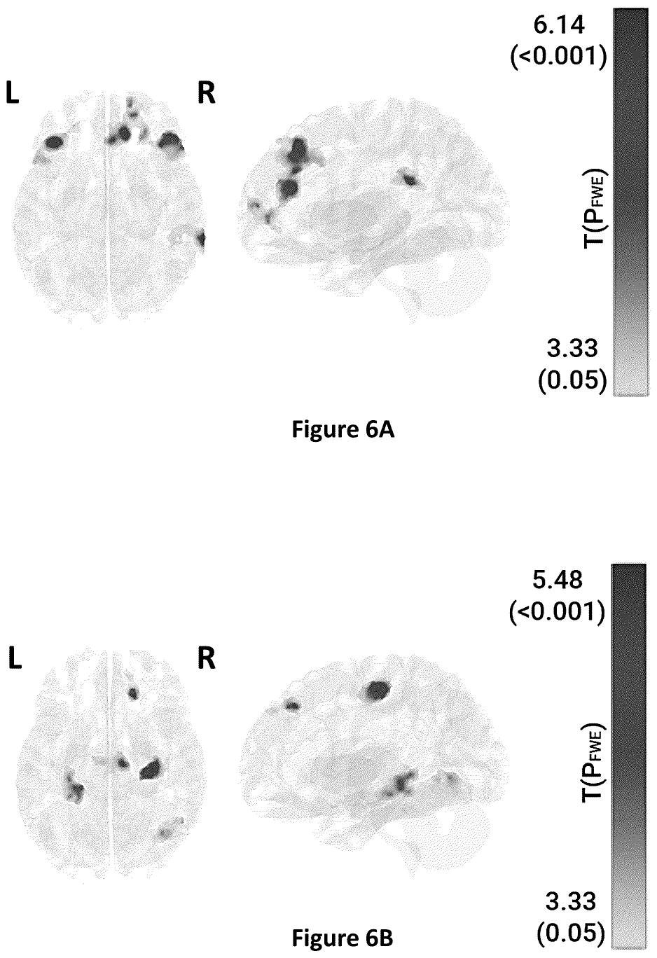

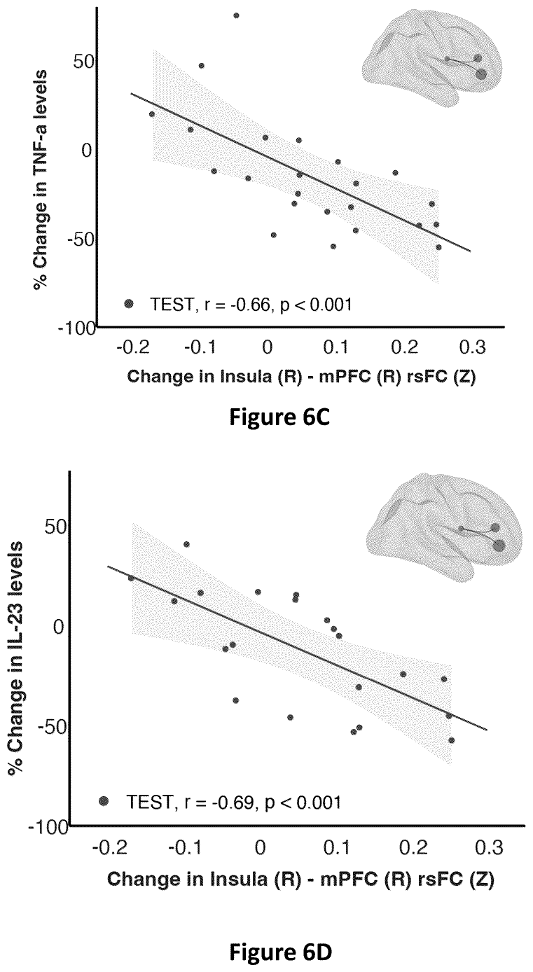

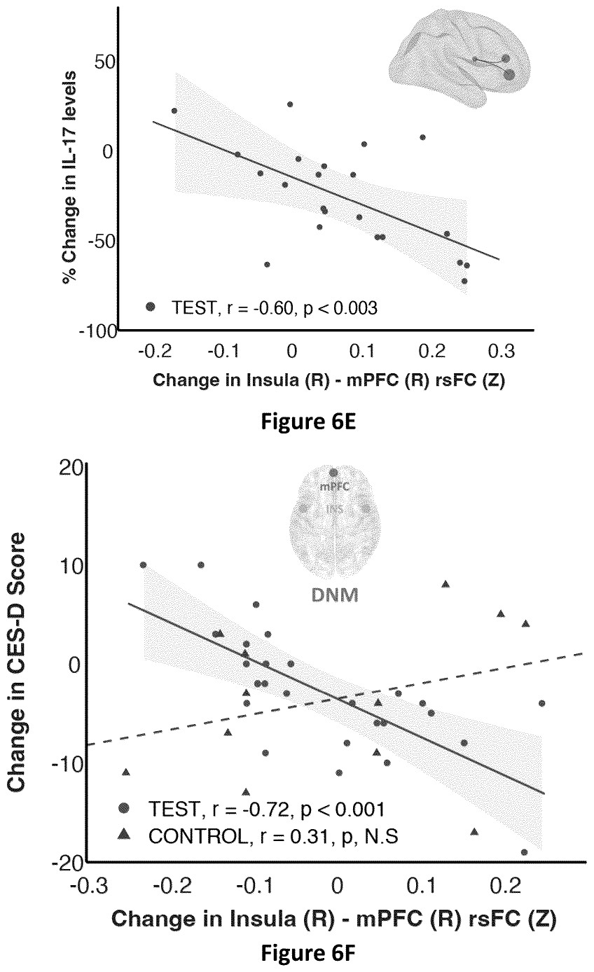

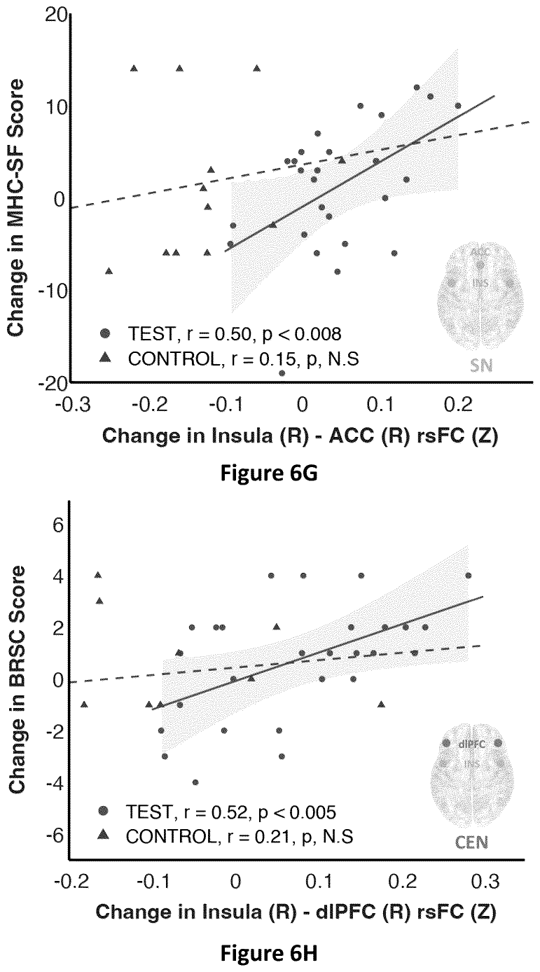

FIGS. 6A-6H show the association between brain plasticity, peripheral inflammation, and psychological state. In FIGS. 6A-6B, regarding brain connectivity: Group-by-time differences in rsFC between insula (R/L) and mPFC (voxel p<0.001, cluster p<0.05, FWE-corrected) suggest insula's key role in the brain-immune axis (n=27). In FIGS. 6C-6E regarding the inflammation link: Increased insula-mPFC rsFC was significantly associated with reduced IL-23, IL-17, and TNF-α levels (voxel p<0.001, FDR-corrected). In FIGS. 6F-6H regarding psychological improvement: rsFC changes in insula-mPFC, ACC, and dlPFC correlated with improved depression, mental health, and resilience scores (voxel p<0.001, FDR-corrected). Thus, the mobile intervention led to significant improvements in psychological state and immune function. Combined behavioural, neural, and immune measures revealed early mechanisms of change, highlighting the role of fronto-limbic circuits, especially the insula and amygdala.

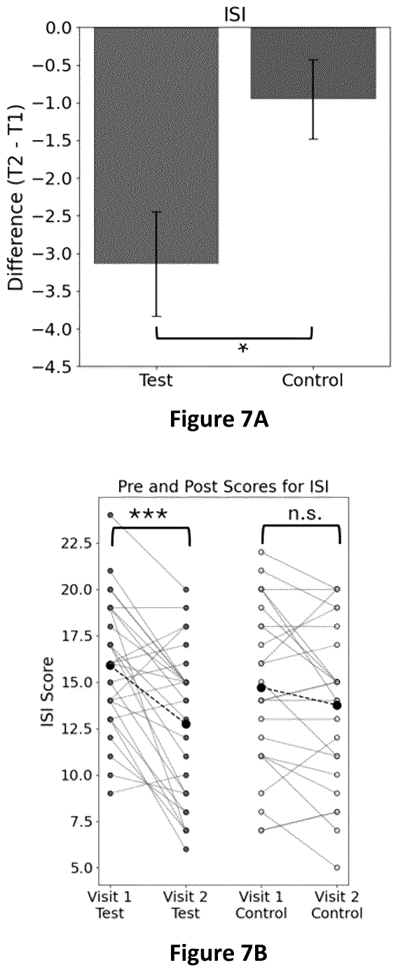

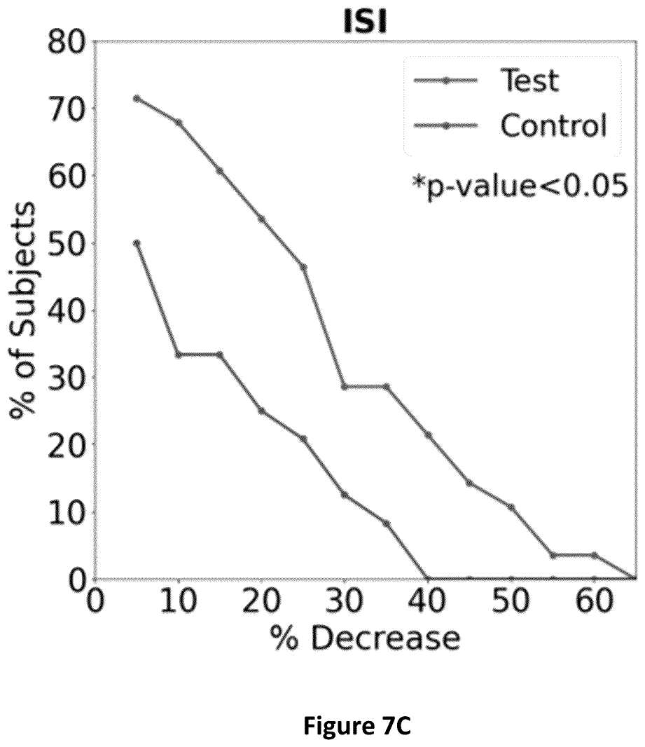

FIGS. 7A-7C show significant improvements in the ISI scores between subject with sleep difficulties. The ISI is a 7-item scale focusing on insomnia symptom severity and impact over the past 2-4 weeks. Items rate difficulty falling asleep, staying asleep, early morning awakening, satisfaction with sleep, interference with daytime functioning, noticeability of impairment, and distress about sleep problems. Thus the digital interventions of the invention improved insomnia symptoms.

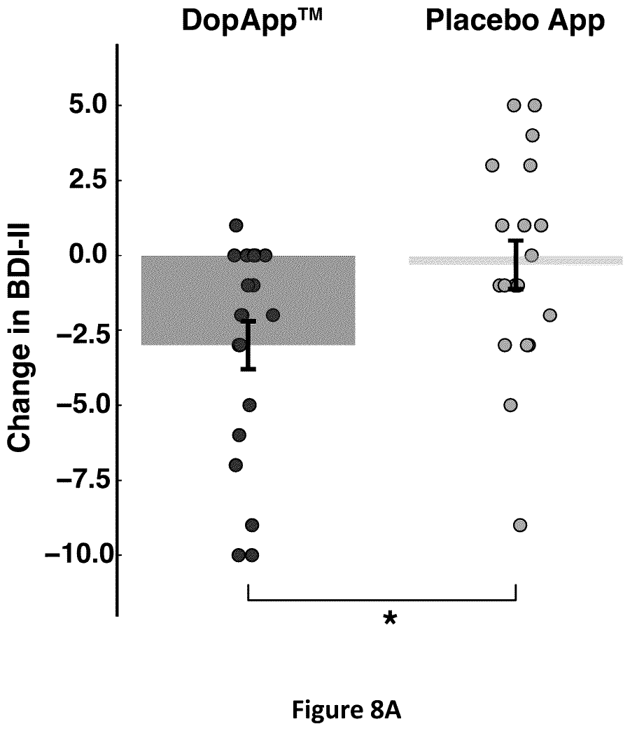

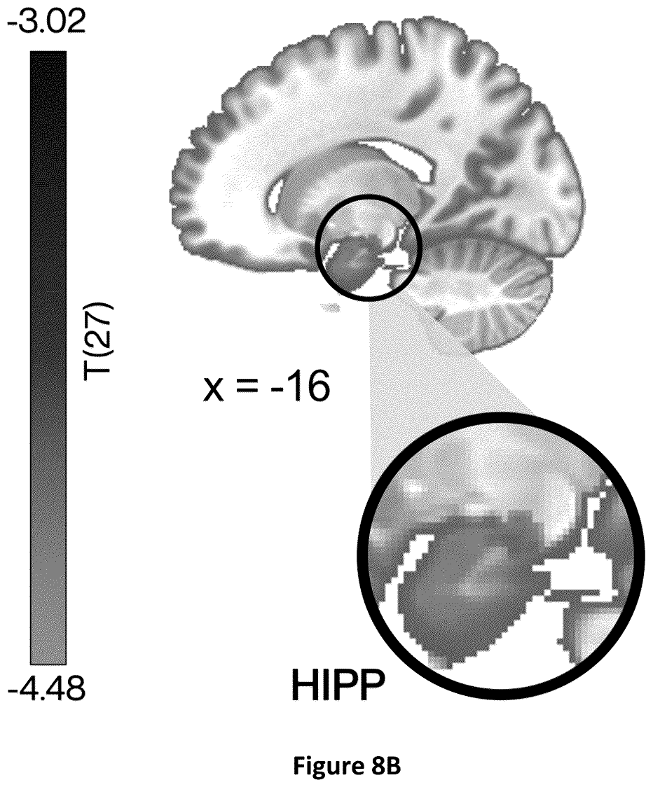

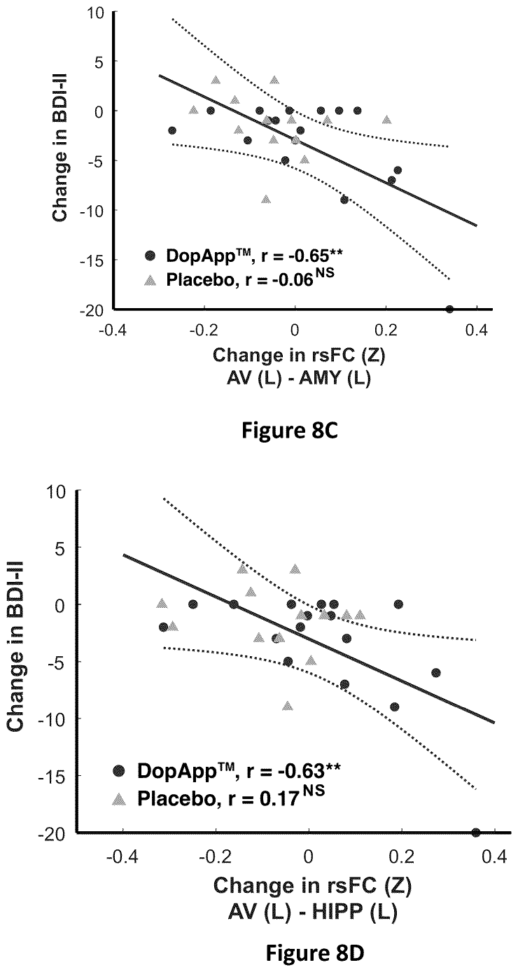

FIGS. 8A-8D shows results from a study which uses the digital interventions of the invention. There was a post intervention improvement in depressive symptoms, Beck's Depression Inventory II (BDI-II) scores. This improvement was correlated with brain connectivity changes within the limbic circuit.

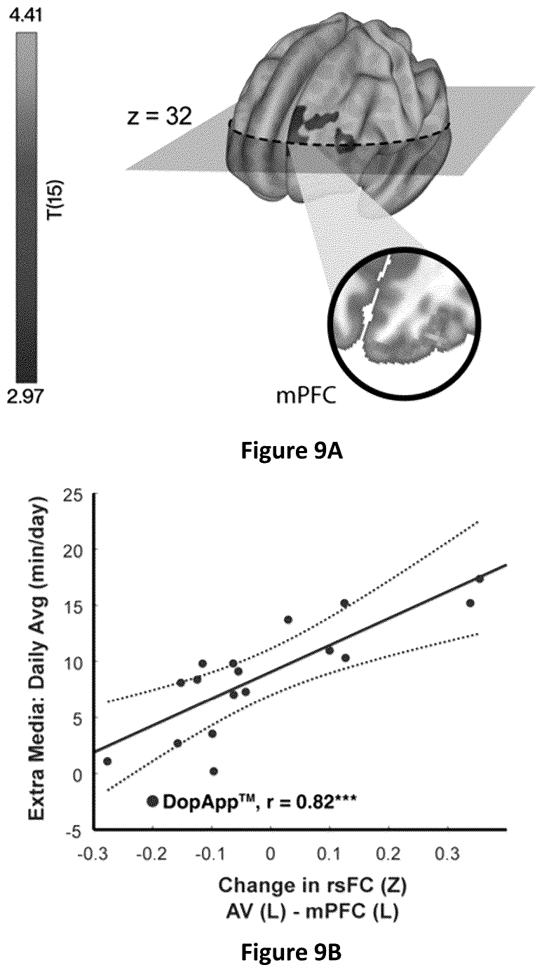

FIGS. 9A-9B show that, regarding app usage, there is an increased rsFC between the left AV and the medial prefrontal cortex was correlated with higher self-initiated daily doses of emotion regulation content usage. This neural pathway, which is part of the DMN, is implicated in self-referential processing and emotional regulation. Thus the software application (also referred to as the digital app, app, or DopApp) use improved depressive symptoms with correlation to brain connectivity changes. Higher self-engagement was correlated with connectivity changes in related brain networks. Engagement with specific app modules predicted corresponding clinical improvements and associated connectivity changes, indicating dose-dependent, and circuit-specific neuroplasticity.

In one embodiment, the term “a” or “one” or “an” refers to at least one. In one embodiment the phrase “two or more” may be of any denomination, which will suit a particular purpose. In one embodiment, “about” or “approximately” may comprise a deviance from the indicated term of +1%, or in some embodiments, −1%, or in some embodiments, ±2.5%, or in some embodiments, ±5%, or in some embodiments, ±7.5%, or in some embodiments, ±10%, or in some embodiments, ±15%, or in some embodiments, ±20%, or in some embodiments, ±25%.

Those skilled in the art to which this invention pertains will readily appreciate that numerous changes, variations, and modifications can be made without departing from the scope of the presently disclosed subject matter, mutatis mutandis.

1. A method for improving reproductive outcomes in a woman in need thereof, the method comprising:
delivering a therapeutic plan to the woman via a personal electronic device, wherein the therapeutic plan comprises therapeutic interventions selected from: at least one physical activity intervention, at least one psychological intervention, and at least one nutrition management intervention;
converting the therapeutic plan into a plurality of targeted interaction outputs for presentation to the woman; and
tracking the woman's responses and associated performance metrics throughout implementation of the therapeutic plan, and continuing until a predetermined condition is met.

2. The method of claim 1 further comprising administering at least one medicinal agent before, during, after, or any combination thereof, the implementation of at least one of the therapeutic interventions.

3. The method of claim 2 wherein the at least one medicinal agent comprises a medicinal agent used in fertility treatment protocols or assisted reproductive technologies.

4. The method of claim 3 wherein the fertility treatment protocols or assisted reproductive technology is selected from: in vitro fertilization protocols, ovulation induction, intrauterine insemination, controlled ovarian stimulation, luteal phase support, or combinations thereof.

5. The method of claim 2 wherein the at least one medicinal agent comprises any of the following selected from: gonadotropins, gonadotropin-releasing hormone (GnRH) agonists, GnRH antagonists, human chorionic gonadotropin (hCG), progesterone, estrogen, letrozole, and clomiphene citrate, or any combination thereof.

6. The method of claim 2 further comprising administering at least one adjunct medicinal agent in combination with the at least one medicinal agent.

7. The method of claim 2 further comprising generating and delivering medication reminders to the woman.

8. The method of claim 2 wherein the therapeutic plan is adapted to a phase of an IVF cycle selected from: suppression phase, ovarian stimulation phase, trigger phase, oocyte retrieval phase, fertilization phase, luteal phase support phase, embryo transfer phase, post-transfer phase, pregnancy testing phase, or any combination thereof.

9. The method of claim 2 further comprising delivering a conditioning stimulus before, during, after, or a combination thereof, the administering of the at least one medicinal agent, at least one of the therapeutic interventions, or a combination thereof.

10. The method of claim 1 further comprising obtaining a plurality of attributes associated with the woman to generate an initial profile for the woman, wherein the therapeutic plan is generated and/or customized based on the initial profile.

11. The method of claim 10 wherein the plurality of attributes are selected from: age, ovarian reserve, hormone profile, reproductive history, prior response to ovarian stimulation, body mass index, genetic factors, comorbidities, medications, nutritional factors, exercise or lifestyle factors, mental wellbeing, inflammatory markers, insulin sensitivity, and patient-defined reproductive goals.

12. The method of claim 10 further comprising:
dynamically monitoring the woman's responses and associated performance metrics during implementation of the therapeutic plan;
generating a dynamic user profile based on the woman's responses and associated performance metrics and other parameters selected from: the initial profile, Electronic Medical Records (EMR) data, psychological attributes, cognitive attributes, pharmacological attributes, feedback of the woman, feedback of a medical practitioner, drug intake parameters, self-reported data, wearable device data, sensor data, and daily device usage patterns, or a combination thereof; and
recursively adapting the therapeutic plan based on the dynamic user profile.

13. The method of claim 12 wherein the recursive adapting of the therapeutic plan comprises dynamically identifying and modifying intervention parameters to implement optimized therapeutic outcomes towards a predetermined therapeutic goal.

14. The method of claim 13 wherein the intervention parameters comprise any of the following selected from: type, timing, dosage, intensity, sequence, duration, frequency, mode of delivery, level of guidance, feedback modality, language adaptation, complexity level, interface configuration, integration with external systems, gamification intensity, or a combination thereof, of one or more of the therapeutic interventions.

15. The method of claim 12 wherein the recursive adapting is performed using a feedback control algorithm, Bayesian inference, machine learning models, AI-driven analytics, or a combination thereof.

16. The method of claim 1 wherein the at least one physical activity intervention comprises at least one low-impact physical activity.

17. The method of claim 16 further comprising monitoring activity level during the at least one physical activity intervention using smartphone sensors, wearable technology, or a combination thereof, and providing personalized guidance based on real-time metrics selected from: step count, movement intensity, user-reported fatigue, distance, time, or a combination thereof.

18. The method of claim 1 wherein the at least one psychological intervention is selected from: heart rate variability (HRV) biofeedback, guided imagery, psychoeducation, cognitive behavioral therapy, stress and anxiety management training, mindfulness-based interventions, body scanning training, sleep hygiene, fatigue training, pain psychology, pacing training, acceptance and commitment therapy (ACT), dialectical behavior therapy (DBT), attention training techniques, psychodynamic therapy, solution-focused brief therapy (SFBT), narrative therapy, gestalt therapy, behavioral activation therapy, adjustment therapy, grief therapy, motivational therapy, meaning-centered therapy, creative therapy, expressive therapy, logotherapy, progressive muscle relaxation, and breathing exercises, or a combination thereof.

19. The method of claim 1 wherein the at least one nutrition management intervention comprises any of the following selected from:
delivering information on a set of dietary supplements and recording the woman's intake of each supplement at specific times relative to meals;
providing instructions for following one or more predetermined diet types, including instructions for substituting specified foods with designated healthier alternatives and preparing meals according to specific recipes;
delivering psychoeducational content that instructs the woman to follow a predetermined anti-inflammatory diet, including instructions for substituting specified foods with designated healthier alternatives and preparing meals according to specific recipes;
delivering dietary guidance configured to stabilize blood glucose levels and maintain insulin sensitivity, including instructions for timing and composition of food intake to maintain blood glucose within a predetermined healthy range;
providing meal logging and nutritional tracking functionality;
generating prompts at predetermined times after meal consumption that instruct the woman to perform the at least one of the therapeutic interventions; and
providing electronic notifications to the woman that prompt selection of designated healthy food options at scheduled eating times;
providing psychoeducational content and interactive guidance configured to reduce stress-related eating behaviors; or
any combination thereof.

20. The method of claim 1 wherein each of the therapeutic interventions has a duration ranging between 10 s to 60 mins.

21. The method of claim 1 wherein the predetermined condition is selected from: a predetermined number of iterations, a predetermined duration has elapsed, a predetermined number of therapeutic interventions have been completed, predetermined progress measurement, predetermined regression measurement, target threshold score achieved, transition to maintenance therapy, no further improvement is measured, achievement of a target follicle count or ovarian response, attainment of a target embryo quality or implantation potential, improvement in hormone levels relevant to reproductive function, improvement in endometrial receptivity markers or thickness, achieving a viable pregnancy, reaching a target threshold score in a fertility outcome metric or clinical scoring system, improvement in stress or psychological assessment scores, and reduction in inflammatory markers, or a combination thereof.

22. The method of claim 1 wherein the targeted interaction outputs are selected from: visual, auditory, tactile, olfactory, thermal, or a combination thereof.

23. The method of claim 1 wherein the improving reproductive outcomes comprises any of the following selected from: improving pregnancy outcomes, improving implantation rates, increasing the number of follicles, improving live birth outcomes, reducing miscarriage, increasing the likelihood that a woman initiates a subsequent treatment cycle following a failed cycle, improving egg quality, and improving embryo quality, or a combination thereof.

24. The method of claim 1 further comprising delivering gamification elements configured to engage the reward system and reinforce adherence to the therapeutic plan, adherence to the at least one medical agent regimen, exploration of additional fertility treatment options, or a combination thereof.

25. A software application embodied on a non-transitory computer-readable medium, the application comprising instructions which, when executed by a processor, cause the processor to perform the method of claim 1.

26. A digital therapeutic system for improving reproductive outcomes in a woman in need thereof, the system comprising:
a data-input module configured to onboard the woman and dynamically monitor and store data related to the woman throughout a therapeutic plan; and
a digital therapy delivery module comprising a processor configured to deliver to the woman a therapeutic plan comprising therapeutic interventions selected from: at least one physical activity intervention, at least one psychological intervention, and a nutrition management intervention;
wherein the digital therapy delivery module is further configured to convert the therapeutic plan into a plurality of targeted interaction outputs for presentation to the woman and track the woman's responses.

27. The system of claim 26 wherein the data-input module is configured to obtain a plurality of attributes associated with the woman to generate an initial profile for the woman, and wherein the processor is further configured to generate and/or customize the therapeutic plan based on the initial profile.

28. The system of claim 26 wherein the digital therapy delivery module comprises software executable on a personal electronic device.

29. The system of claim 26 wherein the digital therapy delivery module is a smartphone.

30. The system of claim 26 wherein the digital therapy delivery module comprises any of the following selected from: a personal electronic device, a graphical user interface, audio interface, haptic interface, olfactory interface, thermal interface, or a combination thereof.

31. The system of claim 26 wherein the data-input module is configured to receive data from a plurality of data acquisition sources in real-time, wherein the data comprises one or more of: Electronic Medical Records (EMR) data, motion sensor data, position sensor data, environmental sensors data, audio sensors data, location sensors data, audio recording data, speech recognition data, gaze-tracking data, touch interaction data, physiological sensor data, optical sensor data, imaging sensors data, self-reported data, device usage pattern data, and camera data, or a combination thereof.

32. The system of claim 26 wherein the processor is further configured to:
dynamically monitor any of the following factors of the woman selected from: responses and associated performance metrics, Electronic Medical Records (EMR) data, motor attributes, psychological attributes, cognitive attributes, pharmacological attributes, and drug intake parameters, self-reported data, wearable device data, device usage patterns, or a combination thereof, throughout the implementation of the therapeutic plan;
generate a dynamic user profile, and recursively adapt the therapeutic plan based on the dynamic user profile, performance metrics, and the woman's responses; or
a combination thereof.

33. The system of claim 26 further comprising at least one additional device operatively coupled to the digital therapy delivery module, selected from: health monitoring systems, medical devices, haptic devices, external speakers, headphones, virtual reality headsets, augmented reality glasses or devices, biofeedback sensors, wearable activity trackers, smartphones, tablets, smartwatches, personal computational devices, smart speakers, voice assistants, motion tracking sensors, virtual assistant systems, internet hubs, gait sensors, eye-tracking devices, voice recording devices, wearable devices, smell-releasing devices, and connected haptic devices, or combinations thereof.

34. The system of claim 26 further comprising a scheduling module configured to generate medication reminders according to each patient's dosage and timing.
